(19) United States
(12) Patent Application Publication
(10) Pub. No.: US 2005/0233994 A1
(43) Pub. Date: Oct. 20, 2005

(54) Methods and vectors for expressing siRNA

(76) Inventors: Ajamete Kaykas, Seattle, WA (US); Randall T. Moon, Kenmore, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/826,130

(22) Filed: Apr. 16, 2004

(51) Int. Cl. A61K 48/00; C12Q 1/68; C12N 15/09

(52) U.S. Cl. 514/44; 435/6; 435/320.1

(57) ABSTRACT

In a first aspect, the invention provides expression vectors comprising: (a) a first RNA polymerase III promoter operably associated with a first RNA polymerase III termination signal and (b) a second RNA polymerase III promoter operably associated with a second RNA polymerase III termination signal, wherein the first and second RNA polymerase III promoters are oriented to promote bidirectional transcription of an insert disposed between the first and the second RNA polymerase III termination signals. In other aspects, the invention provide methods for using these expression vectors for inhibiting expression of target genes, for determining the effect of siRNAs on biological processes, and for identifying siRNAs that affect biological processes.

### FIELD OF THE INVENTION

The present invention is directed to methods and vectors for expressing small interfering RNAs (siRNAs).

### BACKGROUND OF THE INVENTION

RNA interference (RNAi) is an evolutionarily conserved process that functions to inhibit gene expression (Bernstein et al. (2001) Nature 409:363-6; Dykxhoorn et al. (2003) Nat. Rev. Mol. Cell. Biol. 4:457-67). The phenomenon of RNAi was first described in Caenorhabditis elegans, where injection of double-stranded RNA (dsRNA) led to efficient sequence-specific gene silencing of the mRNA that was complimentary to the dsRNA (Fire et al. (1998) Nature 391:806-11). RNAi has also been described in plants as a phenomenon called post-transcriptional gene silencing (PTGS), which is likely used as a viral defense mechanism (Jorgensen (1990) Trends Biotechnol. 8:340-4; Brigneti et al. (1998) EMBO J. 17:6739-46; Hamilton & Baulcombe (1999) Science 286:950-2). Introduction of long dsRNA into a variety of organisms such as Drosophila, Trypanosoma, and pre-implanted mouse oocytes has been shown to specifically inhibit the complementary mRNA (Brigneti et al. (1998) EMBO J. 17:6739-46; Hamilton & Baulcombe (1999) Science 286:950-2; Kasschau & Carrington (1998) Cell 95:461-70). However, in somatic mammalian cells long dsRNA also induces the interferon response, which globally inhibits translation by induction of the kinase, PKR, and 2′, 5′-oligoadenylate synthetase. This has limited the use of RNAi as a tool to study gene function in mammalian cells (Stark et al. (1998) Annu. Rev. Biochem. 67:227-64; Elbashir et al. (2001) Nature 411:494-8).

The first indication that the molecules that regulate PTGS were short RNAs processed from longer dsRNA was the identification of short 21 to 22 nucleotide dsRNA derived from the longer dsRNA in plants (Hamilton & Baulcombe (1999) Science 286:950-2). This observation was recapitulated in Drosophila embryo extracts where long dsRNA was found processed into 21-25 nucleotide short RNA by the RNase III type enzyme, Dicer (Elbashir et al. (2001) Nature 411:494-8; Elbashir et al. (2001) EMBO J. 20:6877-88; Elbashir et al. (2001) Genes Dev. 15:188-200). These observations led Elbashir et. al to test if synthetic 21-25 nucleotide synthetic dsRNAs function to specifically inhibit gene expression in Drosophila embryo lysates and mammalian cell culture (Elbashir et al. (2001) Nature 411:494-8; Elbashir et al. (2001) EMBO J. 20:6877-88; Elbashir et al. (2001) Genes Dev. 15:188-200). They demonstrated that small interfering RNAs (siRNAs) had the ability to specifically inhibit gene expression in mammalian cell culture without induction of the interferon response. These observations led to the development of many techniques for the specific knockdown of genes in mammalian cell culture.

Of these techniques, plasmid-based systems that generate hairpin siRNAs are very appealing (Brummelkamp et al. (2002) Science 296:550-3; Paddison et al. (2002) Genes Dev. 16:948-58; Paddison et al. (2002) Proc. Natl. Acad Sci. U.S.A. 99:1443-8; Paul et al. 2002) Nat. Biotechnol. 20:404-8). These vectors are fairly inexpensive and have been shown to inhibit multiple genes both transiently and in long-term experiments. However, hairpin vectors suffer from multiple limitations. Hairpins can be hard to synthesize in bacteria, difficult to sequence, and the oligonucleotides needed to generate them can be costly and error-prone (Paddison et al. (2002) Proc. Natl. Acad Sci. U.S.A. 99:1443-8; Esposito et al. (2003) Biotechniques 35:914-6, 918, 920). In addition, the hairpin length and sequence can affect the ability of the siRNA to inhibit gene expression (Brummelkamp et al. (2002) Science 296:550-3; Kawasaki & Taira (2003) Nucleic Acids Res. 31:700-7). One of the largest limitations of hairpin vectors is that each strand of the double stranded siRNA is transcribed from different template DNAs. This limits the ability of using hairpin vectors to generate random and cDNA siRNA libraries.

There is a need for expression vectors and methods for expressing siRNAs that circumvent the limitations of hairpin siRNA expression vectors. The present invention addresses this and other needs.

### SUMMARY OF THE INVENTION

In a first aspect, the invention provides expression vectors. The expression vectors comprise: (a) a first RNA polymerase III promoter operably associated with a first RNA polymerase III termination signal; and (b) a second RNA polymerase III promoter operably associated with a second RNA polymerase III termination signal, wherein the first and second RNA polymerase III promoters are oriented to promote bidirectional transcription of an insert disposed between the first and the second RNA polymerase III termination signals. In some embodiments, the expression vectors further comprise a cleavage site for a restriction enzyme disposed within each of the first and second RNA polymerase III termination signals. The expression vectors may comprise a recognition site for a restriction enzyme, wherein the cleavage site for the restriction enzyme is located outside the recognition site for the restriction enzyme. The recognition site is typically within the first and second RNA polymerase III promoters. Exemplary restriction enzymes that cleave outside their recognition site comprise Alw1, Bbs1, Bbv1, BceA1, BciV1, BfuA1, Bmr1, Bpm1, BpuE1, Bsa1, BseR1, Bsg1, BsmA1, BsmB1, BsmF1, BspM1, Ear1, Eci1, Fau1, Fok1, Hga1, Hph1, MboII, Mly1, Mnl1, Ple1, Sap1, and SfaN1. In some embodiments, the restriction enzyme is BsmB1.

The vectors of the invention may further comprise an insert disposed between the first and second RNA polymerase III termination signals. The size of the insert is generally between 19 an 29 nucleotides, such as between 19 and 23 nucleotides, such as 19 nucleotides. The vectors of the invention may be plasmid vectors, viral vectors, or linear vectors, and may also comprise selectable markers and/or an origins of replication operable in a eukaryotic cell.

In some embodiments, the first aspect of the invention provides a plurality of expression vectors, each comprising: (a) a first RNA polymerase III promoter operably associated with a first RNA polymerase III termination signal; (b) a second RNA polymerase III promoter operably associated with a second RNA polymerase III termination signal, wherein the first and second RNA polymerase III promoters are oriented to promote bidirectional transcription of an insert disposed between the first and the second RNA polymerase III termination signals; and (c) an insert disposed between the first and second RNA polymerase III termination signals.

In a second aspect, the invention provides methods for inhibiting expression of a target gene. The methods comprise introducing into a host cell an expression vector comprising: (a) a first RNA polymerase III promoter operably associated with a first RNA polymerase III termination signal; (b) a second RNA polymerase III promoter operably associated with a second RNA polymerase III termination signal; and (c) a target gene-specific insert disposed between the first and the second RNA polymerase III termination signals, wherein the first and second RNA polymerase III promoters are oriented to promote bidirectional transcription of the target gene-specific insert to produce siRNA molecules that inhibit the expression of a target gene. The size of the insert is generally between 19 an 29 nucleotides, such as between 19 and 23 nucleotides, such as 19 nucleotides.

In a third aspect, the invention provides methods for determining the effect of an siRNA on a biological process. The methods comprise the steps of:

(a) introducing into one or more host cells an expression vector comprising:
(i) a first RNA polymerase III promoter operably associated with a first RNA polymerase III termination signal;
(ii) a second RNA polymerase III promoter operably associated with a second RNA polymerase III termination signal; and
(iii) an insert disposed between the first and the second RNA polymerase III termination signals, wherein the first and second RNA polymerase III promoters are oriented to promote bidirectional transcription of the insert to produce siRNA molecules; and

(b) determining the effect of the siRNA molecules on a biological process of the one or more host cells. The size of the insert is generally between 19 an 29 nucleotides, such as between 19 and 23 nucleotides, such as 19 nucleotides. The insert may comprise a random sequence of oligonucleotides.

Biological processes according to this aspect of the invention include, but are not limited to, biological processes that mediate biological signal transduction pathways, expression of a cell surface molecules, and stem cell differentiation. The effect on the biological process may be determined using a reporter gene. In some embodiment, step (a) comprises introducing a plurality of expression vectors into one or more cells, wherein substantially all the vectors comprise a different insert. The methods may further comprise the step of identifying at least one insert from which siRNA molecules are transcribed that produce the effect on the biological process.

In a fourth aspect, the invention provides methods for identifying an siRNA that affects a biological process. The methods comprisethe steps of:

(a) introducing a plurality of expression vectors comprising a plurality of inserts into one or more cells, wherein each of the plurality of expression vectors comprises:
(i) a first RNA polymerase III promoter operably associated with a first RNA polymerase III termination signal;
(ii) a second RNA polymerase III promoter operably associated with a second RNA polymerase III termination signal; and
(iii) an insert disposed between the first and the second RNA polymerase III termination signals, wherein the first and second RNA polymerase III promoters are oriented to promote bidirectional transcription of the insert to produce siRNA molecules; and

(b) identifying at least one insert from which siRNA molecules are transcribed that affect a biological process of the one or more cells.

All, or substantially all, of the expression vectors may comprise a different insert.

In a fifth aspect, the invention provides kits for creating expression vectors for producing siRNA molecules. In some embodiments, the kits comprise:

(a) an expression vector comprising:
(i) a first RNA polymerase III promoter operably associated with a first RNA polymerase III termination signal;
(ii) a second RNA polymerase III promoter operably associated with a second RNA polymerase III termination signal; and
(iii) a restriction enzyme cleavage site disposed within each of the first and second RNA polymerase III termination signals, wherein the first and second RNA polymerase III promoters are oriented to promote bidirectional transcription of an insert introduced between the restriction enzyme cleavage sites; and

(b) packaging.

In some embodiments, the kits comprise:

(a) a first primer for amplifying a sense strand of a nucleic acid molecule comprising a first RNA polymerase III promoter operably associated with a first RNA polymerase III termination signal;

(b) a second primer for amplifying an antisense strand of a nucleic acid molecule comprising a second RNA polymerase III promoter operably associated with a second RNA polymerase III termination signal;

(c) a double-stranded nucleic acid template comprising the first RNA polymerase III promoter or the second RNA polymerase III promoter; and

(d) packaging.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A shows a diagram of an expression vector according to the invention, as described in EXAMPLE 1. The expression vector (pHippy) has convergent opposing human H1 and U6 polymerase III promoters that drive expression of both strands of any template cloned in between the BsmB1 cloning sites. The expression vector also contains the pUC origin or replication and the Zeocin-resistance gene for propagation and replication in bacteria. As depicted the H1 and U6 promoters contain a polymerase III termination signal (TTTTT) between the −5 to −1 position of the promoter, and BsmB1 recognition sites. BsmB1 is a type II restriction enzyme, which cuts outside of its recognition sequence. In the expression vector shown, BsmB1 cleavage leaves 3′ TTTT overhangs on both strands of the plasmid, as depicted. Inserts consisting, for example, 19 nucleotides, can be cloned into the expression vector as double-stranded oligonucleotides by addition of AAAA to the 5′ ends of the oligonucleotides, as depicted. FIG. 1B shows exemplary inserts that can be cloned into the expression vector.

### DETAILED DESCRIPTION OF THE PREFERRED EMBODIMENT

Unless specifically defined herein, all terms used herein have the same meaning as they would to one skilled in the art of the present invention.

The first aspect of the invention provides expression vectors. The expression vectors of the invention comprise: (a) a first RNA polymerase III promoter operably associated with a first RNA polymerase III termination signal, and (b) a second RNA polymerase III promoter operably associated with a second RNA polymerase III termination signal, wherein the first and second RNA polymerase III promoters are oriented to promote bidirectional transcription of an insert disposed between the first and the second RNA polymerase III termination signals.

As used herein, the term “expression vector” or “vector” refers to any nucleic acid construct that is adapted for expressing siRNA molecules. A vector according to the invention may or may not include an insert that will be transcribed to produce siRNA molecules. The vectors of the invention may be linear or circular and include plasmids, cosmids, viruses (bacteriophage, animal viruses, plant viruses), and artificial chromosomes. Suitable vectors may be shuttle vectors such that they are capable of being reproduced in large amounts in prokaryotic or eukaryotic systems and then introduced into host cells.

The vectors according to the invention may be vectors that are capable of integrating into the genome of a cell. Exemplary integrating vectors include, but are not limited to, retroviral vectors, such as the pBABE vectors, lentiviral vectors, adeno-associated virus (AAV) vectors, or plasmids. Alternatively the vector may be one that is capable of replicating as an extrachromosomal element such as an artificial chromosome or an Epstein Barr-based virus.

An “expression cassette” refers to a linear vector according to the invention comprising the regulatory sequences for expressing siRNA molecules and an insert that can be transcribed to produce siRNA molecules.

The vectors of the invention include a first and a second RNA polymerase III promoter. In nature, RNA polymerase III promoters are responsible for the expression of a variety of genes, including H1 RNA genes, 5S RNA genes, U6 RNA genes, adenovirus VA1, Vault, telomerase RNA genes, tRNA genes, Epstein-Barr-virus-encoded RNAs (EBER), and human 7SL RNA genes. There are three types of RNA polymerase III promoters. In types I (e.g., 5S RNA genes) and II (e.g., tRNA genes), the promoter elements are within the transcribed regions, whereas in type III genes (e.g., H1 and U6 RNA genes), the promoter elements are found only in the 5′ flanking region (reviewed in Paule & White (2000) Nucl. Acids Res. 28:1283-1298, which publication is incorporated herein by reference). Type III RNA polymerase III promoters have three elements: a TATA element generally located at about −35 to about −25 from the transcriptional start site, a proximal sequence element (PSE) generally located at about −70 to about −45, and a distal sequence element (DSE) generally located at about −260 to about −190, although it can be closer, such as between −95 and −80 in the H1 gene (see, e.g., Myslinki et al. (2001) Nucleic Acids Res. 2001 29(12):2502-9, incorporated herein by reference). The PSE is quite variable and binds a protein complex called SNAPc or PTF. The consensus sequence for PSE is 5′ tnaccntnant/cnnaaagt/ag 3′ (SEQ ID NO:1) (Boyd et al. (1995) J. Mol. Biol. (1995) 253: 677-90, incorporated herein by reference). The DSE is also quite variable and usually consists of any combination of (1) an octamer motif that binds the transcriptional activator Oct-1, (2) binding sites for the transcriptional activator Staf, and binding sites for the transciptional activator Sp1. As used herein, the term “RNA polymerase III promoter” refers to a type III RNA polymerase III promoter. Any type III RNA polymerase III promoter may be used in the present invention to promote the synthesis of siRNA molecules, such as RNA polymerase III promoters of human or mouse origin, or from any other species, as well as functional derivatives thereof. A functional derivative of an RNA polymerase III promoter includes any synthetic or modified promoter that is able to promote transcription by RNA polymerase III. Such functional derivatives may comprise combinations of the various elements known to be important in RNA polymerase III promoters. Typically, a functional derivative of an RNA polymerase promoter for use in the vectors of the invention comprises a TATA element, a PSE, and at least part of a DSE, with an appropriate spacing between these elements. RNA polymerase III promoters may be modified to be inducible, for example by small molecules such as tetracycline or IPTG (see, e.g., Ohkawa & Taira (2000) Human Gene Therapy 11:577-85; Meissner et al. (2001) Nucl. Acids. Res. 29:1672-82). Such inducible RNA polymerase III promoters may be expressed ubiquitously or in a tissue or temporally specific manner upon induction. RNA polymerase III promoters may also be modified to comprise restriction enzyme recognition sites, as further described below. Exemplary functional derivatives of RNA polymerase III promoters suitable for use in the vectors of the invention include the modified H1 and U6 promoters described in EXAMPLE 1, and whose sequence is provided in SEQ ID NO:2 and SEQ ID NO:3, respectively.

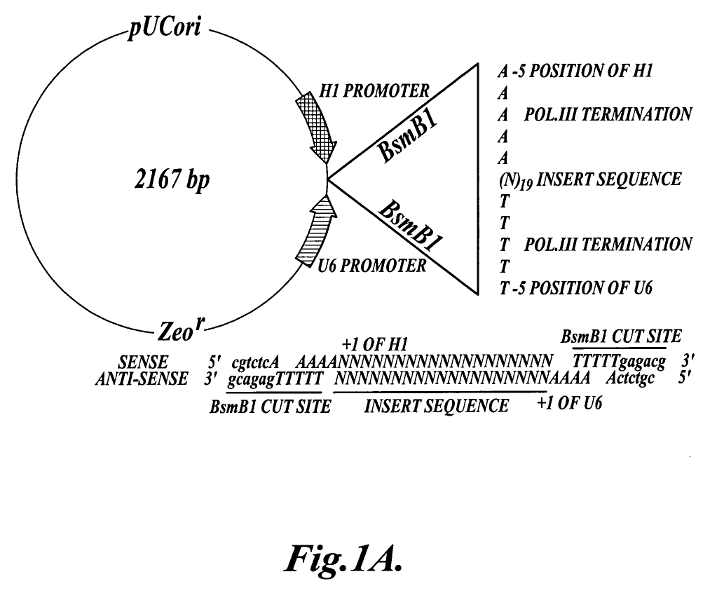

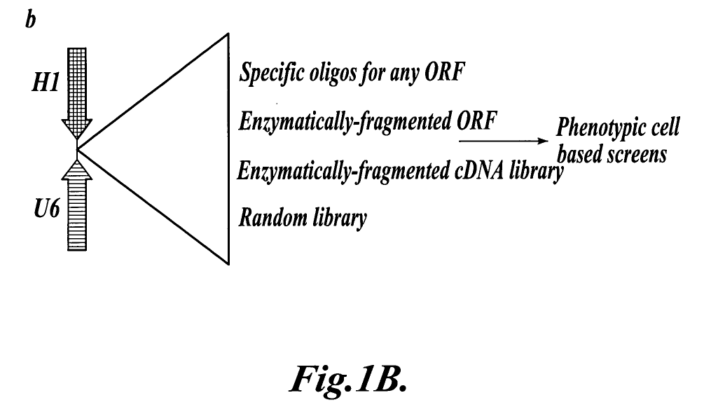

To circumvent generating an inverted repeat, which can cause instability of vectors in bacteria or cells, the first RNA polymerase III promoter may be different than the second RNA polymerase III promoter. For example, the first RNA polymerase III promoter may be a human H1 promoter and the second RNA polymerase III promoter may be a human U6 promoter, as shown in FIG. 1A and described in EXAMPLE 1, or vice versa.

In the vectors of the invention, the first RNA polymerase III promoter is operably associated with a first RNA polymerase III termination signal and the second RNA polymerase III promoter is operably associated with a second RNA polymerase III termination signal. The term “operably associated” refers to the functional relationship between two nucleic acid sequences. For example, a promoter is operably associated to a transcriptional termination signal if it is positioned to so that transcription from that promoter is terminated by the transcriptional termination signal.

Typically, the RNA polymerase III termination signal comprises a series of consecutive thymidines, such as five consecutive thymidines, in the sense strand of the vector. The advantage of such a RNA polymerase III termination signal is that the transcript initiated by an RNA polymerase III promoter such as the U6 or H1 promoter normally terminates after the second or third thymidine of the RNA polymerase termination signal to give rise to a transcript ending with two or three consecutive uridines. These uridines can form the 3′ overhangs in the siRNA necessary for optimal activity. The cleavage site and hence the overhang generated may vary depending on the type of RNA polymerase III promoter used, and the particular system will be chosen to give rise to the overhang of choice, which will typically be two uridine residues.

In the vectors of the invention, the first and second RNA polymerase III promoters are oriented to promote bidirectional transcription of an insert disposed between the first and second RNA polymerase III termination signals, as shown in FIG. 1A and described in EXAMPLE 1. Thus, the first and second RNA polymerase III promoters are operably associated with the insert to transcribe both strands of the insert. The term “insert” as used herein refers to any nucleotide sequence introduced into the vectors of the invention that serves as a template for the expression of siRNA molecules. The size of the insert is generally between 19 and 29 nucleotides, such as between 19 and 25 nucleotides, such as 19 nucleotides. The nucleotide sequence of the insert may be random (i.e., not pre-defined) or it may be defined by the nucleotide sequence of a gene it is targeted to inhibit. Typically, the insert is immediately downstream of the transcriptional start sites for the first and second RNA polymerase III promoters, or separated by a minimal distance such as less than twenty base pairs, preferably less than ten base pairs, even more preferably less than five base pairs, and still more preferably by two or less base pairs. Similarly, the insert is generally immediately upstream of the first and second RNA polymerase III termination signals, or separated by a minimal distance. In some embodiments, the RNA polymerase III promoters, RNA polymerase III termination signals, and the insert are operably associated in such a way that from each strand only the insert and two or three thymidines of an RNA polymerase III termination signal are transcribed.

In some embodiments, the invention provides a plurality of expression vectors, each comprising: (a) a first RNA polymerase III promoter operably associated with a first RNA polymerase III termination signal; (b) a second RNA polymerase III promoter operably associated with a second RNA polymerase III termination signal, wherein the first and second RNA polymerase III promoters are oriented to promote bidirectional transcription of an insert disposed between the first and the second RNA polymerase III termination signals; and (c) an insert disposed between the first and second RNA polymerase III termination signals.

In some embodiments, the vectors of the invention additionally include a cleavage site for a restriction enzyme disposed within each of the first and second RNA polymerase III termination signals. According to these embodiments, cleavage of circular vectors with the restriction enzyme produces two ends that are incompatible for religation. Thus, inserts with ends that are compatible to the ends of the digested vector can be easily cloned with minimal vector religation. Generally, the recognition site for the enzyme is located outside the cleavage site, such as within the first and second RNA polymerase III promoters. For example, in some embodiments of the vectors of the invention, the first and second RNA polymerase III promoters have been modified to include a restriction enzyme recognition site. Exemplary restriction enzymes that cleave outside of their recognition site include, but are not limited to, Alw1, Bbs1, Bbv1, BceA1, BciV1, BfuA1, Bmr1, Bpm1, BpuE1, Bsa1, BseR1, Bsg1, BsmA1, BsmB1, BsmF1, BspM1, Ear1, Eci1, Fau1, Fok1, Hga1, Hph1, MboII, Mly1, Mnl1, Ple1, Sap1, and SfaN1. The recognition sites for restriction enzymes are described, for example, in New England BioLabs Catalog & Technical Reference (2002-03), which publication is incorporated herein by reference. In some embodiments, the first and second RNA polymerase III promoters include a restriction enzyme recognition site for BsmB1, as shown in FIG. 1A. Exemplary modified H1 and U6 RNA polymerase III promoters including a restriction enzyme recognition site for BsmB1 are provided in SEQ ID NO: 2 and SEQ ID NO:3, respectively.

Vectors according to the invention may include various selection markers and/or reporter genes. These may be used for selection in the bacterial system the plasmids are grown in, but also for selection of transfected cells. Examples of reporter genes which may be employed to identify transfected cell lines include alkaline phosphatase (AP), beta galactosidase (LacZ), beta glucoronidase (GUS), chloramphenicol acetyltransferase (CAT), green fluorescent protein (GFP), horseradish peroxidase (HRP), and luciferase (Luc). Exemplary antibiotic selectable markers include those that confer resistance to ampicillin, bleomycin, chloramphenicol, gentamycin, hygromycin, kanamycin, lincomycin, methotrexate, phosphinothricin, puromycin, zeocin, and tetracyclin. The vectors of the invention may also include hybrid selection marker/reporter genes, such as a zeocin/GFP hybrid gene.

Exemplary methods for constructing the expression vectors of the invention are described in EXAMPLES 1-3. The construction of vectors of the invention is easier and less expensive than the construction of siRNA hairpin vectors. Moreover, the vectors of the invention can be generated in a high-throughput manner using the polymerase chain reaction (PCR) without propagation through bacteria, as described in EXAMPLE 2. Moreover, the use of the vectors of the invention to produce siRNAs results in increased levels of inhibition of specific gene expression compared to equivalent siRNA hairpin vectors, as shown in EXAMPLE 1.

The vectors of the invention may be used, for example, to determine the function of known genes by inhibiting the expression of such genes, as shown in EXAMPLE 1 and further described below. The vectors of the invention may also be used to generate siRNA expression libraries (from inserts with random and/or defined sequences) that can be used in screening assays, as described in EXAMPLE 3. siRNA expression libraries may be expressed from inserts with randon and/or defined sequences. For example, libraries of siRNAs could be produced by fragmenting one or more cDNA libraries and inserting the fragments into the vectors of the invention, or by inserting random oligonucleotide libraries into the vectors of the invention. Accordingly, the vectors of the invention may be used for phenotypic screens, genome-wide target identification, and functional genomics. Other uses for the vectors of the invention include, but are not limited to, modulation of the level of expression of genes, validation of potential drug targets, and therapeutic applications. Therapeutic applications include, but are not limited to, the treatment of infectious conditions (e.g., hepatitis virus or human immunodeficiency virus infections) by administering vectors of the invention to inhibit expression of infectious agents, and the treatment of any conditions associated with genes that are over-expressed or mis-expressed (e.g., cancers or immune disorders) by administering vectors of the invention to inhibit expression of such genes. The vectors of the invention may also be used to create transgenic organisms, such as transgenic mice, using methods standard in the art (see, for example, Carmell et al. (2003) Nature Struct. Biol. 10:91-2, incorporated herein by reference). Transgenic animals comprising the vectors of the invention provide useful model systems, for example, for studying gene function and associated pathologies, for screening candidate drugs effective to treat conditions associated with genes that are over-expressed or mis-expressed, and for drug target validation (see, e.g., Aza-Blank et al. (2003) Mol. Cell. 12:627-37; Zheng et al. (2003) Proc. Natl. Acad Sci. U.S.A. 101:135-40; Brummelkamp et al. (2003) Nature 424: 797-801; Bems et al. (2004) Nature, in press, which publications are incorporated herein by reference).

In a second aspect, the invention provides methods for inhibiting expression of a target gene. The methods comprise the step of introducing into a host cell an expression vector comprising (a) a first RNA polymerase III promoter operably associated with a first RNA polymerase III termination signal; (b) a second RNA polymerase III promoter operably associated with a second RNA polymerase III termination signal; and (c) a target gene-specific insert disposed between the first and the second RNA polymerase III termination signals, wherein the first and second RNA polymerase III promoters are oriented to promote bidirectional transcription of the target gene-specific insert to produce siRNA molecules that inhibit the expression of a target gene.

According to the second aspect of the invention, an expression vector is used to inhibit the expression of a target gene. The term “target gene” refers to any gene whose expression it is desired to inhibit using the vectors of the invention. The purpose of the inhibition may be therapeutic, for example, or to study the function of the target gene. The target gene may be chromosomal or extrachromosomal. The target gene may be endogenous to the cell or it may be a foreign gene, such as a transgene. Typically, the target gene is a eukaryotic gene, but alternatively the target gene may be a viral, bacterial, fungal, or protozoan gene expressed in a eukaryotic host cell. The target gene may be a protein-coding gene or a gene that does not encode a protein, such as a gene that codes for ribosomal RNAs, splicosomal RNA, tRNA, or other a structural or enzymatic RNA. In some embodiments the target gene may be a gene family comprising a conserved sequence. The target gene may also be a specific allele of a gene, such as a mutant allele, or a splice variant. Target gene-specific inserts may be selected in silico by screening appropriate databases for unique nucleotide sequences of a suitable size. Exemplary methods for selecting target gene-specific inserts in silico are described in EXAMPLE 1.

Any gene expressed in a host cell may be a target gene. Exemplary target geness include, but are not limited to, genes involved in signal transduction (e.g., kinases, kinase inhibitors, cyclins, phosphatases, etc.), genes involved in growth and differentiation (e.g., cyclins, adhesion molecules, transforming growth factor-beta family members, Wnt family members, Hox family members, Pax family members, cytokines or lymphokines and their receptors, oncogenes, etc.), and genes encoding enzymes (e.g., dehydrogenases, reverse transcriptases, lipases, ATPases, DNA and RNA polymerases, etc.).

Exemplary expression vectors comprising a first and second RNA polymerase III promoter operably associated with a first and second RNA polymerase III termination signal, respectively, and oriented to promote bidirectional transcription of an insert disposed between the first and second RNA polymerase III termination signals to produce siRNA molecules are as described for the first aspect above. The expression vectors used according to the second aspect of the invention comprise a target gene-specific insert that is transcribed to produce siRNA molecules. The term “target gene-specific insert” refers to an insert whose nucleotide sequence is defined by the nucleotide sequence of the target gene it is intended to inhibit. Typically, the nucleotide sequence of the target gene-specific insert is identical to the nucleotide sequence of a region within the target gene. In some embodiments, the target gene-specific insert is specific for a conserved sequence in a family of genes. Thus, the target gene-specific insert may be used to inhibit the expression of more than one gene. A target gene-specific insert may also be used to inhibit expression of a particular allele of a gene, such as a mutant allele associated with a disorder. The size of the insert is generally between 19 and 29 nucleotides, such as between 19 and 25 nucleotides, such as 19 nucleotides

The expression vectors comprising a target gene-specific insert are introduced into a host cell. The term “host cell” refers to any cell derived from or contained in any organism, such as a plant, animal, protozoan, virus, bacterium, or fungus. The host cell may be a germ line cell or a somatic cell, totipotent or pluripotent, dividing or non-dividing, undifferentiated (such as a stem cell) or differentiated, a primary cell or an immortalized cell, an abnormal cell (e.g., a mutant cell) or a normal cell. Host cells may comprise, but are not limited to, blood cells (such as hematopoietic progenitor cells and stem cells, lymphocytes, macrophages, and other blood lineage cells), bone marrow cells, brain cells, blood vessel cells, liver cells, lung cells, breast cells, cartilage cells, corneal cells, endometrial cells, endothelial cells, kidney cells, muscle cells, pancreatic cells, neurons, glia, colon cells, skin cells, or epithelial cells. Host cells also may comprise cells within organisms, such as plants and animals. The plant may be a monocot, dicot, or gymnosperm. The animal may be a vertebrate or invertebrate. Examples of vertebrates include fish, and mammals, such as cattle, goat, pig, sheep, hamster, mouse, rat, and human; example of invertebrates include nematodes, insects, arachnids, and other arthropods. In some embodiments, the host cell is a mammalian cell.

The vectors of the invention may be introduced into host cells or organisms in vitro, in vivo, or ex vivo using a variety of methods known in the art, including but not limited to, transfection (transient or stable), transduction, lipofection, electroporation, microinjection, jet injection (e.g., for intra-muscular delivery as described, for example, in Furth et al. (1992) Anal. Biochem. 205:365-8, incorporated herein by reference), particle bombardment (as described, for example, in Tang et al. (1992) Nature 356:152-4, incorporated herein by reference), enteral or parenteral delivery (e.g., oral, buccal, anal, vaginal, pulmonary, intravenous, intra-arterial, intramuscular, intraperitoneal, topical, transdermal, intradermal, intraperitoneal, subcutaneous, or other appropriate routes), and hydrodynamic nucleic acid administration protocols (described, for example, in Chang et al. (2001) J. Virol. 75:3469-73; Liu et al. (1999) Gene Ther. 6:1258-66; Wolff et al. (1990) Science 247:1465-8; Zhang et al. (1999) Hum. Gene Ther. 10:1735-7; which publications are incorporated herein by reference). For example, the vectors of the invention may be delivered in vivo as a viral vector or as a linear expression cassette. Successful delivery of siRNA-producing vectors in vivo has been demonstrated using various methods (reviewed for example in Dorsett & Tuschl (2004) Nat. Rev. Drug. Discovery 3:318-29, incorporated herein by reference. See also Xia et al. (2002) Nat. Biotechnol. 20:1006-10; Arts et al. (2003) Genome Res. 13:2325-32; Hommel et al. (2003) Nature Med. 9:1539-44; Rubinson et al. (2003 Nature Genet. 33:401-6; van de Wettering et al. (2003) EMBO Rep. 4:609-15; Matsudo & Cepko (2003) Proc. Natl. Acad. Sci. U.S.A. 101:16-22; Kong et al. (2004) EMBO Rep. 5:183-8; Song et al. (2003) Nature Med. 9:347-51; Sullinger & Gilboa (2002) Nature 418:252-8; Opalinska & Gewirtz (2002) Nature Rev. Drug Discov. 1:503-14; which publications are incorporated herein by reference).

The vectors of the invention may be incorporated into a variety of formulations for therapeutic delivery. For example, the vectors of the invention may be formulated into pharmaceutical compositions by combination with appropriate, pharmaceutically acceptable carriers, diluents, additives, lubricants, buffering agents, etc. Exemplary formulations include, but are not limited, to preparations in solid, semi-solid, liquid, or gaseous forms, such as tablets, capsules, powders, granules, ointments, solutions, emulsions, suppositories, inhalents, and aerosols (see Remington's Pharmaceutical Sciences, Mack Publishing Co., Eastern Pa., 17th ed, (1985), incorporated herein by reference). The determination of an effective amount or dose of the vectors of the invention is well within the capability of those skilled in the art. Thus, the amount actually administered will be dependent upon the individual subject, and will preferably be an optimized amount such that the desired effect is achieved without significant side-effects.

In the methods of the second aspect of the invention, bidirectional transcription of the target gene-specific insert in the expression vectors produces siRNA molecules that inhibit the expression of the target gene. “Inhibit the expression of a target gene” refers to the reduction of the level of target gene expression or the elimination of expression of the target gene, compared to the expression of the target gene in a host cell that does not contain the expression vector with the target gene-specific insert. The level of expression of the target gene may be monitored by examining RNA expression and/or protein expression. The consequences of expressing target gene-specific siRNA molecules may be assessed by examination of the outward properties of the host cell or organism, or using techniques to monitor target gene RNA and/or protein expression, such as RNA solution hybridization, Northern analysis, reverse transcription, in situ hybridization, monitoring expression using microarrays, antibody binding, immunocytochemistry, Western blotting, enzyme-linked immunoassays, radioimmunoassays, other immunoassays, and fluorescence-linked cell analysis. Target gene expression may be assayed using reporter genes whose product is easily monitored, such as alkaline phosphatase, beta-galactosidase, green fluorescent protein, horseradish peroxidase, luciferase, etc. When the target gene is a mutant allele, the consequences of expressing target gene-specific siRNA molecules may be assessed using methods that discriminate between the expression of the wild-type allele and the mutant allele, such as single-strand conformational polymorphisms, denaturing gel electrophoresis, allele-specific PCR, or antibodies capable of discriminating between the two alleles. Quantitation of the level of target gene expression allows the efficiency of the inhibition to be determined.

Typically, the level of inhibition of the target gene is at least 5%, for example at least 10%, at least 20%, at least 30%, at least 40%, at least 50%, or at least 60% of the uninhibited level of expression of the target gene in the host cell. That is, if the level of inhibition of the target gene is 10%, the target gene is reduced by 10% such that it is expressed at 90% of the uninhibited level of its expression in the host cell. The level of inhibition may be in excess of 60%, such as in excess of 75%, in excess of 90%, or in excess of 95% of the uninhibited level of expression of the target gene. In some embodiments, the level of inhibition is, or almost is, 100%, and hence the host cell or organism will in effect have the phenotype equivalent to a so-called “knock out” of the target gene. However, in some embodiments it may be desirable to achieve only partial inhibition so that the phenotype is equivalent to a so-called “knock down” of the target gene. A knock-down phenotype may be advantageous, for example, for target genes that are required for cell survival. Partial inhibition of expression of such target genes may allow for cell survival and analysis of gene function. The level of inhibition may be selected, for example, by adjusting the amount of the expression vector introduced into the host cell or by using different inserts specific for the same target gene, as described in EXAMPLE 1.

In a third aspect, the invention provides methods for determining an effect on a biological process. The methods comprise the steps of:

(a) introducing into one or more host cells an expression vector comprising:
(i) a first RNA polymerase III promoter operably associated with a first RNA polymerase III termination signal;
(ii) a second RNA polymerase III promoter operably associated with a second RNA polymerase III termination signal; and
(iii) an insert disposed between the first and the second RNA polymerase III termination signals, wherein the first and second RNA polymerase III promoters are oriented to promote bidirectional transcription of the insert to produce siRNA molecules; and

(b) determining the effect of the siRNA molecule on a biological process of the one or more host cells.

Exemplary expression vectors comprising a first and second RNA polymerase III promoter operably associated with a first and second RNA polymerase III termination signal, respectively, and oriented to promote bidirectional transcription of an insert disposed between the first and second RNA polymerase III termination signals to produce siRNA molecules are as described for the first aspect above. The expression vectors used according to the third aspect of the invention comprise an insert that is transcribed to produce siRNA molecules. Typically, the size of the insert is between 19 and 29 nucleotides, such as between 19 and 25 nucleotides, such as 19 nucleotides. The insert may have a predefined sequence. For example, the insert may be specific for a target gene of previously unknown function. Expression vectors comprising inserts specific for a target gene of previously unknown function may be used to determine the function of such a gene. In some embodiments, the insert comprises a random sequence. Expression vectors comprising random sequence inserts may be used to obtain an effect on one or more biological process. The sequence of the insert may also be partly predefined and partly random.

In one of the steps of the methods, the expression vectors are introduced into one or more host cells. The host cells used in this aspect of the invention, and methods of introducing the expression vectors into the host cells, are as described above.

Another step of the methods comprises determining an effect on a biological process. The term “biological process” refers to any process occurring in or between one or more host cells. Thus, biological processes include, but are not limited to, signal transduction, growth, proliferation development, differentiation, metabolism, disease resistance, cell division, secretion, transcription, translation, splicing, cell-cell communication, endocytosis, exocytosis, antigen presentation, cell death, and the like. The biological process may be an abnormal (mutant or pathological) process. For example, the methods of the invention may be used to determine an effect on a pathological biological process, such as modifying or abolishing the pathological process. The term “determining an effect” refers to any measurable qualitative or quantitative effect on a biological process in the one or more host cells or their progeny. The effect may be measured, for example, as a morphological, biochemical, physiological, molecular, cellular, behavioral effect on the level of single cells, collection of cells, tissues, organs, or whole organisms.

In some embodiments of the third aspect of the invention, the methods comprise introducing a plurality of expression into one or more host cells, wherein all or substantially all of the plurality of expression vectors comprise a different insert. For example, the expression vectors may comprise a library of different inserts. In some embodiments, the methods further comprise the step of identifying at least one insert from which siRNA molecules are transcribed that affects a biological process. Generally, the insert is identified by sequencing the insert contained in the one or more host cells exhibiting the effect on the biological process using standard methods in the art. In some embodiments, the expression vector comprising the insert from which siRNA molecules are transcribed that affect a biological process is isolated from the one or more host cells before sequencing the insert.

In a fourth aspect, the invention provides methods for identifying an siRNA that produces an effect on a biological process. The methods comprise the steps of:

(a) introducing a plurality of expression vectors comprising a plurality of inserts into one or more host cells, wherein each of the plurality of expression vectors comprises:
(i) a first RNA polymerase III promoter operably associated with a first RNA polymerase III termination signal;
(ii) a second RNA polymerase III promoter operably associated with a second RNA polymerase III termination signal; and
(iii) an insert disposed between the first and the second RNA polymerase III termination signals, wherein the first and second RNA polymerase III promoters are oriented to promote bidirectional transcription of the insert to produce siRNA molecules; and

(b) identifying at least one insert molecule from which siRNA molecules are transcribed that affect a biological process of the one or more host cells.

Exemplary expression vectors comprising a first and second RNA polymerase III promoter operably associated with a first and second RNA polymerase III termination signal, respectively, and oriented to promote bidirectional transcription of an insert disposed between the first and second RNA polymerase III termination signals to produce siRNA molecules are as described for the first aspect above. The expression vectors used according to the fourth aspect of the invention each comprise an insert that is transcribed to produce siRNA molecules, as described for the third aspect above.

In one of the steps of the methods, a plurality of expression vectors are introduced into one or more host cells. Generally, all or substantially all of the plurality of expression vectors comprise a different insert. For example, about 75%, about 80%, about 90%, or about 95% of the vectors may comprise a different insert. Accordingly, the plurality of expression vectors may comprise a library of different inserts. Exemplary methods for preparing a plurality of expression vectors comprising different inserts are described in EXAMPLE 3. The host cells used in this aspect of the invention, and methods of introducing the expression vectors into the host cells, are as described above.

Another step of the methods comprises identifying at least one insert from which siRNA molecules are transcribed that affect a biological process in the one or more host cells. Methods of determining an effect on a biological process in one or more host cells are as described in the third aspect of the invention. Generally, the insert(s) from which siRNA molecules are transcribed that affect a biological process in the one or more host cells is identified by sequencing the insert inside the one or more host cells exhibiting the effect on the biological process using standard methods in the art. In some embodiments, the expression vector comprising the insert from which siRNA molecules are transcribed that affect a biological process is isolated from the cells before sequencing the insert.

In a fifth aspect, the invention provides kits for creating an expression vector for producing siRNA molecules. In some embodiments the kits comprise:

(a) an expression vector comprising:
(i) a first RNA polymerase III promoter operably associated with a first RNA polymerase III termination signal;
(ii) a second RNA polymerase III promoter operably associated with a second RNA polymerase III termination signal; and
(iii) a restriction enzyme cleavage site disposed within each of the first and second RNA polymerase III termination signals, wherein the first and second RNA polymerase III promoters are oriented to promote bidirectional transcription of an insert introduced between the restriction enzyme cleavage sites; and

(b) packaging.

Exemplary expression vectors comprising a first and second RNA polymerase III promoter operably associated with a first and second RNA polymerase III termination signal, respectively, and oriented to promote bidirectional transcription of an insert disposed between the first and second RNA polymerase III termination signals to produce siRNA molecules, and a restriction enzyme cleavage site disposed within each of the first and second RNA polymerase III termination signals, are as described for the first aspect above. The expression vectors may be packaged in aqueous media or in lyophilized form. Exemplary packaging includes at least one container, such as a vial, tube, bottle, or other suitable container means, into which an expression vector may be placed. The kits of the invention may further comprise instructions for employing the expression vectors as well as other reagents not included in the kit, such as instructions for introducing inserts of interest into the expression vectors.

In further embodiments, the kits comprise:

(a) a first primer for amplifying a sense strand of a nucleic acid molecule comprising a first RNA polymerase III promoter operably associated with a first RNA polymerase III termination signal;

(b) a second primer for amplifying an antisense strand of a nucleic acid molecule comprising a second RNA polymerase III promoter operably associated with a second RNA polymerase III termination signal;

(c) a double-stranded nucleic acid template comprising the first RNA polymerase III promoter or the second RNA polymerase III promoter; and

(d) packaging.

Exemplary primers for creating the expression vectors of the invention are described in EXAMPLES 1 and 3. According to some embodiments, the first primer may comprise a sequence for amplifying the sense strand of nucleic acid molecule comprising a modified H1 promoter, as shown for example in SEQ ID NO:30; the second primer may comprise a sequence for amplifying the antisense strand of a nucleic acid molecule comprising a modified U6 promoter, as shown for example in SEQ ID NO:32; and the double-stranded nucleic acid template may comprise the modified U6 promoter, as described in EXAMPLE 3. Exemplary packaging for the components of the kits is described above. The kits may also comprise instructions, such as instructions for preparing a third primer for amplifying an antisense nucleic acid molecule comprising a 3′ region of the first RNA polymerase III promoter, a 5′ region of the second RNA polymerase III promoter, and an insert disposed between the 3′ region of the first RNA polymerase III promoter and the 5′ region of the second RNA polymerase III promoter; and/or instructions for using the first, second, and third primers to amplify an expression vector comprising the insert, wherein the first and second RNA polymerase III promoters are oriented to promote bidirectional transcription of the insert for producing siRNA molecules.

### EXAMPLES

### Example 1

This example describes the construction of siRNA expression vectors according to the invention by cloning and their use to specifically inhibit target gene expression.

Construction of phippy vector: The pHippy vector contains two opposing RNA polymerase III promoters to drive the expression of both strands of a template DNA cloned in between the promoters. To circumvent generating an inverted repeat, which can cause plasmid instability in E. coli, the human H1 and human U6 polymerase III promoters were used instead of two H1 or two U6 promoters. Both the H1 and U6 promoters were modified to contain a five thymidine polymerase III termination sequence at the −5 to −1 position, and a BsmB1 restriction enzyme recognition site at the −12 to −6 position, as shown in FIG. 1A. The sequence of the modified H1 promoter is provided in SEQ ID NO:2; the sequence of the modified U6 promoter is provided in SEQ ID NO:3. pHippy also contains a PUC origin of replication and the Zeocin-resistance gene as a selectable marker.

The modified U6 promoters were generated by polymerase chain reaction (PCR) from human genomic DNA using Advantage taq (Clontech) with the following primers: 5′ ccccagtggaaagacgcgca 3′ (U6p1, SEQ ID NO:4) and 5′ tttttgagacgctagccacaagatatataaagccaagaaat 3′ (U6p2, SEQ ID NO:5). The PCR product was cloned into PGEM-T (Promega). The modified H1 promoter was synthesized by annealing two oligonucleotides to generate the 97 nucleotide H1 promoter: 5′ atttgcatgtcgctatgtgttctgggaaatcaccataaacgtgaaatgtctttggatttgggaatcttataagtggatcctgagaccgt ctcaaaaa 3′ (H1 promoter, SEQ ID NO:2).

pHippy was generated by blunt ligation of the H1 and U6 PCR products after phosphorylation of the products. The ligated product was PCR amplified with the following primers containing Mlu1 and Not1 restriction sites and cloned into the Mlu1 and Not1 sites of SvZeo: 5′ gaattcgcggccgcatttgcatgtcgctatgt 3′ (H1not1, SEQ ID NO:6) and 5′ gaattcacgcgtccccagtggaaagacgcgca 3′ (U6m1u1, SEQ ID NO:7). SvZeo was constructed by ligating a PCR product containing the pUC origin from pUC18 and an SvZeo expression cassette from pCDNA5/TO (Clontech), using the following primers were used to generate that construct: 5′ gaattcacgcgtgcggccgcccactgagcgtcagaccccgt 3′ (pucori(S), SEQ ID NO:8), 5′ gaattcgccaggaaccgtaaaaaggcc 3′ (pucori(AS), SEQ ID NO:9), 5′ gaattcggatccacgcgtgaatgtgtgtcagttagggt 3′ (SZPA(S), SEQ ID NO:10), and 5′ gaattcggatccgagccccagacatgataagataca 3′ (SZPA(AS), SEQ ID NO:11).

The modifications to the H1 and the U6 promoter described above do not appear to affect the ability of either the H1 of the U6 promoter to promote expression of RNA or the transcriptional start site. The BsmB1 restriction enzyme cleaves the DNA five nucleotides downstream from its recognition site. Digestion of pHippy with BsmB1 produces 3′ overhangs of four thymidines at −1 to −4 of both the modified U6 and the modified H1 promoter and, therefore, renders the two ends incompatible for self-ligation producing a very low level of background ligation.

Cloning inserts into pHippy vectors: Inserts for producing siRNAs were designed using the web based siRNA design program from the Whitehead Institute web page (http://jura.wi.mit.edu/pubint/http://iona.wi.mit.edu/siRNAext/home.php). In general, inserts for producing siRNAs were used from previously published siRNAs information or were designed by introducing the corresponding sequence for a given gene into the Whitehead siRNA design program using the following pattern AAN₁₉TT or AAGN₁₇CN₂. The output sequences for a given gene that were not complimentary to other genes after blasting were chosen, and the corresponding oligonucleotides were designed and ordered.

Oligonucleotides were based on the Whitehead output sequences and modified for cloning into pHippy by addition of 2 or 4 adenines to the 5′ end of the sense and antisense versions of the Whitehead output sequences.

A phippy vector containing an insert specific for PGL3 luciferase was prepared using a previously reported synthetic sequence (Elbashir et al. (2001) Nature 411:494-8). Two oligonucleotides, 5′ aaaaggctcctcagaaacagctc 3′ (PGL3luciferase sense, SEQ ID NO: 12) and 5′ aaaagagctgtttctgaggagcc 3′ (PGL3luciferase antisense, SEQ ID NO:13) were annealed and ligated into pHippy digested with BsmB1. A pHippy vector containing an insert specific for EGFP was prepared by ligating two oligonucleotides, 5′ aaaagcaagctgaccctgaagttcat 3′ (EGFP sense, SEQ ID NO: 14) and 5′ aaaaatgaacttcagggtcagcttgc 3′ (EGFP antisense, SEQ ID NO: 15) pHippy digested with BsmB1. Five pHippy vectors containing an insert specific for Low-density Lipoprotein Receptor-related Protein 6 (LRP6) were prepared by ligating five pairs of oligonucleotides into pHippy digested with BsmB1:

(LRP6#1 sense, SEQ ID NO:16)

5′ aaaaaggttcccttccacatcct 3′
and

(LRP6#1 antisense, SEQ ID NO:17)

5′ aaaaaggatgtggaagggaacct 3′,

(LRP6#2 sense, SEQ ID NO:18)

5′ aaaaaaggttcccttccacatccttt 3′
and

(LRP6#2 antisense, SEQ ID NO:19)

5′ aaaaaaggatgtggaagggaaccttt 3′,

(LRP6#3 sense, SEQ ID NO:20)

5′ aaaagaagatggcagccagggct 3′
and

(LRP6#3 antisense, SEQ ID NO:21)

5′ aaaaagccctggctgccatcttc 3′,

(LRP6#4 sense, SEQ ID NO:22)

5′ aaaaggcacttacttccctgcaa 3′
and

(LRP6#4 antisense, SEQ ID NO:23)

5′ aaaattgcagggaagtaagtgcc 3′,

(LRP6#5 sense, SEQ ID NO:24)

5′ aaaaaaggcacttacttccctgcaatt 3′
and

(LRP6#5 antisense, SEQ ID NO:25)

5′ aaaaaattgcagggaagtaagtgcctt 3′.

More than 25 double-stranded oligonucleotides have been cloned into pHippy with a cloning efficiency of almost 100%. An insert sequence specific for any mRNA can be cloned into pHippy and the DNA insert will be transcribed from both strands and form double-stranded RNA with 5′ overhangs of two uridines, which closely resembles functional siRNA produced by Dicer (Elbashir et al. (2001) Genes Dev. 15:188-200; Brummelkamp et al. (2002) Science 296:550-3; Zamore et al. (2000) Cell 101:25-33). This vector can be used to generate siRNA specific for any target gene, as shown in FIG. 1B. Pools of siRNAs specific for a target gene can be generated by enzymatic digestion of a target gene cDNA to produce small fragments, followed by cloning of the small fragments into pHippy, as shown in FIG. 1B. This strategy circumvents the need to first identify which gene-specific sequences are able to produce functional siRNAs. In addition, libraries of siRNA molecules can be generated by cloning enzymatic digests of cDNA libraries or random oligonucleotide sequences into pHippy. The siRNA libraries can be introduced into cells and populations of cells screened for phenotypic changes. Cells with the desired phenotypic changes can be isolated, allowing the siRNA vector to be rescued and characterized. This type of screen presents an unbiased means to identify genes involved in diverse biological processes.

Construction of hairpin vector for PGL3 luciferase: U6 and H1 hairpin vectors for PGL3 luciferase were generated by PCR amplification of the U6 and H1 promoters. The following primers were used for U6: 5′ tggaaagacgcgcaggca 3′ (U6PGL3p1, SEQ ID NO:26) and 5′ aaaaagagctgtttctgaggagcctctcttgaaggctcctcag aaacagctcggagatctttttgagacgctagccacaa 3′ (U6PGL3p2, SEQ ID NO:27). The following primers were used for HI: 5′ atttgcatgtcgctatgtgt 3′ (H1PGL3p1, SEQ ID NO:28) and 5′ aaaaagagctgtttctgaggagcctctcttgaaggctcctcagaaacagctcggagatctttttgagacggtctcagga 3′ (H1PGL3p2, SEQ ID NO:29). The PCR products were cloned into pGEM-T.

Cell culture and transfections: 293T cells were grown in DMEM supplemented with 10% FBS and 1% Pen/Strep under standard conditions. All transfections were performed in 24 well plates with Lipofectamine Plus or 2000 (Invitrogen) according to the manufacturer's specifications.

Luciferase assays: Luciferase assays were preformed according to the Dual luciferase assay specifications (Promega). In all cases, 293T cells were transfected with 10 nanograms of CMV-PLG3luciferase and 100 picograms of pRLCMV (Promega), and the cells were harvested 24 hours later and assayed for luciferase activity in 96 well plate in a Berthold 96V luminometer. Super(8×)Topflash reporter assays were performed as described (Bernstein et al. (2001) Nature 409:363-9). 293T cells seeded in 24 well plates were transfected with 10 nanograms of Super(8×)Topflash, 100 picograms of pRLCMV (Promega), and the indicated amount of effector plasmids. The concentrations of all transfections were brought up to a total of 250 nanograms with the vector CS2+. Assays were performed as described (Bernstein et al. (2001) Nature 409:363-9) and according to the Dual luciferase assay specifications (Promega).

Confocal microscopy: 2.5×10⁵ 293T cells were seeded in 12 well plates and transfected with 10 nanograms of pEGFPN1 (Clontech), pDSREDN1 (Clontech), and 30 nanograms of the indicated siRNA expression vector. Twenty-four hours after transfection the cells were removed from the plates with PBS and live cells were sandwiched between coverslips and glass slides and visualized for fluorescence using the appropriate lasers and filters to visualize EGFP and DSRED.

pHippy efficiently inhibits the ectopic expression of reporter genes: To determine whether pHippy produces functional siRNAs, a pHippy vector (pHippyPGL3luc) was generated using a PGL3 luciferase-specific insert that had been previously used to produce siRNA directed against PGL3 luciferase (Elbashir et al. (2001) Nature 411:494-8). As controls, hairpin siRNA vectors were generated using the same insert sequence specific for PGL3 luciferase driven from either the U6 (U6PGL3lucHP) or the H1 (H1PLG3lucHP) promoter. To determine the efficiency of inhibition of luciferase by the siRNA vectors, 293T cells were transfected with a cocktail of PGL3 luciferase, Renilla luciferase, pHippy, and U6PGL3lucHP, H1PLG3lucHP, or pHippyPGL3luc. After normalization for transfection with Renilla luciferase, the empty pHippy vector gave similar luciferase levels as cells transfected with luciferase alone, and this level was set to 100% luciferase activity (˜100,000 relative light units), as shown in Table 1, which shows average normalized PGL3 luciferase levels an standard deviations for three experiments.

TABLE 1
Luciferase Assays to Measure the Efficiency of the pHippy Vector

| Plasmid transfected | PGL3luc Activity (%) |
|---|---|
| pHippy | 100 |
| 30 nanograms U6PGL3lucHP | 32 +/− 2 |
| 30 nanograms H1PGL3lucHP | 19 +/− 1.5 |
| 1 nanogram pHippyPGL3luc | 48 +/− 4 |
| 3 nanograms pHippyPGL3luc | 44 +/− 3 |
| 10 nanograms pHippyPGL3luc | 18 +/− 1 |
| 30 nanograms pHippyPGL3luc | 10 +/− 0.5 |
| 100 nanograms pHippyPGL3luc | 3 +/− 1 |
| 100 nanograms pHippyEGFP | 95 +/− 10 |

Both of the vectors that express hairpin RNAs against PGL3 luciferase significantly inhibited luciferase expression. The pHippy vector with the insert specific for PGL3 luciferase also inhibited expression of PGL3 luciferase. Moreover, at the same concentration of DNA transfected, pHippyPGL3luc is more efficient at inhibiting Luciferase expression than either of the two hairpin vectors. Specifically, at 30 nanograms of vector, transfected PHippyPGL3luc is 2-4 times more efficient than inhibition of PGL3 luciferase by the vectors that generate hairpin RNAs. A likely explanation for the increased efficiency of pHippy might be because hairpin RNAs have to be processed by Dicer, while siRNA expressed by pHippy would already be functional without further processing.

To determine whether pHippy generates sequence-specific inhibitory siRNAs, a pHippy vector was generated containing an insert specific for EGFP. pHippyEGFP did not inhibit the expression of PGL3 Luciferase (Table 1). However, it inhibited the expression of EGFP as assayed by confocal microscopy. This not only establishes that the inhibition of expression of EGFP and Luciferase is gene-specific, but also demonstrates that pHippy can theoretically be used to knock down the expression of any gene. Supportingly, the expression of more than 10 unique genes have been inhibited using pHippy. On average, three to four constructs with unique inserts have to be tested to obtain expression of functional siRNA, which is similar to other siRNA systems. pHippy efficiently inhibits the expression of endogenous genes: To determine whether pHippy can be used to inhibit the expression of endogenous genes, five pHippy constructs were generated against the human Low-density Lipoprotein Receptor-related Protein 6 (LRP6). To first demonstrate that the constructs could inhibit expression of ectopic LRP6, they were screened for their ability to inhibit expression of a fusion protein consisting of LPR6 and Renilla luciferase (LRP6Rluc). This fusion of a target gene to Renilla luciferase allows for rapid quantitative assessment of the efficiency of any given siRNA construct. 293T cells were co-transfected with the constructs and assayed for luciferase activity 48 hours later. The level of luciferase expression in cells transfected with LRP6Rluc and pHippy was set to 100%. All experiments were normalized for transfection with PGL3 luciferase. The average normalized Renilla luciferase activities and standard deviations for three experiments are shown in Table 2. Three of the five pHippy constructs generated against LRP6 inhibited LRP6Rluc expression by more than 50%, as shown in Table 2.

TABLE 2
Inhibition of Ectopic LRP6 Expression by
pHippy Vectors With LRP6-Specific Inserts

| Plasmid transfected | LRP6Rluc Activity (%) |
|---|---|
| pHippy | 100 |
| 30 nanograms pHippyEGFP | 98 |
| 30 nanograms pHippyLRP6#1 | 82 |
| 30 nanograms pHippyLRP6#2 | 33 |
| 30 nanograms pHippyLRP6#3 | 120 |
| 30 nanograms pHippyLRP6#4 | 18 |
| 30 nanograms pHippyLRP6#5 | 13 |

To test whether expression of endogenous LRP6 was also inhibited by the pHippy constructs, an indirect readout of the biological function of LRP6 was measured. Briefly, LRP6 is part of the Wnt receptor signaling complex, and it is required to receive and transduce Wnt signaling to down-stream components in the Wnt signaling cascade (Schweizer & Varmus (2003) BMC Cell Biol. 4:4). Thus, the biological function of LRP6 culminates in the activation of β-catenin-mediated transcription, which can be efficiently measured by activation of the Super(8×)Topflash reporter (Veeman et al. (2003) Curr. Biol. 13:680-5). To test whether LRP6 is required for Wnt signal transduction in human cells, 293T cells were co-transfected with a cocktail of Super(8×)Topflash, Renilla luciferase, and the five pHippy constructs with LRP5-specific inserts or control pHippy constructs. 24 hours after transfection, the cells were treated with Wnt3a-conditioned media (Wnt3a-CM) to activate Wnt signaling, and thus the expression of Super(8×)Topflash, then cultured for an additional 24 hours. After normalization for transfection with Renilla luciferase, the cells transfected with empty pHippy or pHippyEGFP were set to 1 fold activation (˜10,000 relative light units), as shown in Table 3.

TABLE 3
Inhibition of Endogenous LRP6 Expression by
pHippy Vectors With LRP6-Specific Inserts

| | Fold Activation |
|---|---|
| Plasmid transfected | of SuperTopflash |
| pHippy | 1 |
| 30 nanograms pHippyEGFP | 1 |
| 30 nanograms pHippyLRP6#1 | 2 |
| 30 nanograms pHippyLRP6#2 | 4 |
| 30 nanograms pHippyLRP6#3 | 1 |
| 30 nanograms pHippyLRP6#4 | 1.5 |
| 30 nanograms pHippyLRP6#5 | 1 |
| pHippy + Wnt3a-conditioned medium (Wnt3a-CM) | 102 |
| 30 nanograms pHippyEGFP + Wnt3a-CM | 95 |
| 30 nanograms pHippyLRP6#1 + Wnt3a-CM | 110 |
| 30 nanograms pHippyLRP6#2 + Wnt3a-CM | 51 |
| 30 nanograms pHippyLRP6#3 + Wnt3a-CM | 108 |
| 30 nanograms pHippyLRP6#4 + Wnt3a-CM | 37 |
| 30 nanograms pHippyLRP6#5 + Wnt3a-CM | 21 |

Treatment with Wnt3a increases the reporter activation about 100-fold, as shown in Table 3. Cotransfection of some of the pHippyLPR6 constructs inhibited Wnt3a activation of Super(8×)Topflash more than 50% (Table 3). Strikingly, there was a strict correlation between the ability of the pHippyLRP6 constructs to inhibit the expression of LRP6Rluc and the ability to inhibit Wnt3a activation of Super(8×)Topflash (Tables 2 and 3). These sets of experiments demonstrate that pHippy constructs have the ability to inhibit expression and thus the biological function of endogenous genes.

### Example 2

This example describes the construction of siRNA expression vectors according to the invention by PCR and their use to specifically inhibit target gene expression.

To generate pHippy siRNA constructs in a rapid manner, a PCR method was devised that incorporates the U6 and H1 promoters from pHippy on either end of a PCR product. A gene-specific primer for a target gene can be sandwiched between the two convergent promoters. To develop this system three oligonucleotides were synthesized:

(1) a 97 nucleotide primer consisting of the entire modified H1 promoter from pHippy, 5′ atttgcatgtcgctatgtgttctgggaaatcaccataaacgtgaaatgtctttggatttgggaatcttataagtggatcctgagaccgt ctcaaaaa 3′ (H1p97, SEQ ID NO:30);

(2) a target gene-specific primer containing 18 nucleotides of complimentary sequence to both the modified H1 and U6 promoters and 21 nucleotides of gene-specific (PGL3 luciferase) or random control sequences, 5′ ctgagaccgtctcaaaaa ggctcctcagaaacagctc tttttgagacgctagcca 3′ (H1-PGL3-U6, SEQ ID NO: 31); and

(3) an 18 nucleotide anti-sense primer to the modified human U6 promoter, 5′ TGGAAAGACGCGCAGGCA 3′(U6p3, SEQ ID NO:32).

pHippy siRNA expression cassettes were generated by a single step multiple primer PCR. In short, 10 nanograms of plasmid containing the human U6 promoter was used as template for PCR in a 50 microliter reaction containing 2 microliters of 10 pm/microliter U6 primer (SEQ ID NO:32), microliters of the primer encompassing the entire H1 promoter (SEQ ID NO:30), 2 microliters of 0.01 pm/microliters of the gene-specific linker primer (e.g., SEQ ID NO:31), 10 microliters of 2 mM dNTPs, 10 microliters of advantage buffer, and 0.5 microliters tag-advantage (Clontech). The PCR products were generate by 30 cycle of touchdown PCR program that ramped down from 60° C to 50° C.

After PCR with the H1 primer, the U6 primer, and limiting amounts of the gene-specific primer using a U6 template, a single robust band of the appropriate size was generated for all of the different gene-specific primers tested. These PCR products were cleaned with a nucleospin column and co-transfected into 293T cells with the PGL3 luciferase reporter and Renilla luciferase as a normalization control, as described in EXAMPLE 1. Neither of the control PCR products significantly inhibited PGL3 luciferase activity, as shown in Table 4.

TABLE 4
Luciferase Assays to Measure Inhibition by PCR Vectors

| Plasmid or PCR Product Transfected | Luciferase Activity (%) |
|---|---|
| pHippy | 100 |
| 100 nanograms PCR control 1 | 100 +/− 10 |
| 100 nanograms PCR control 2 | 98 +/− 10 |
| 30 nanogram pHippyPGL3luc | 8 +/− 2 |
| 10 nanograms PCR PGL3luc | 85 +/− 8 |
| 30 nanograms PCR PGL3luc | 52 +/− 5 |
| 100 nanograms PCR PGL3luc | 36 +/− 4 |

However, the PCR product specific for PGL3 luciferase (PCR PGL3luc) inhibited luciferase in a dose-dependent manner (Table 4). Although PCR PGL3luc inhibited PGL3 luciferase expression, it was not as efficient as the pHippyPGL3luc. This may be due to faster degradation of the PCR product and might be circumvented by additional sequences on the 5′ and 3′ ends of the PCR product.

### Example 3

This example describes the construction of siRNA expression vectors according to the invention for transcribing random libraries of siRNA molecules.

The pHippy system is well suited for generation of cDNA or random insert libraries because both strands of DNA template are transcribed to generate siRNA. To determine whether pHippy could in principle be used for a random screen, a random library of sequences based on PGL3 luciferase was generated. This library was generated by randomizing the final 3 nucleotides (CTC) in the sense strand of the PGL3-specific insert described in EXAMPLE 1 and corresponds to a library of 64 possible inserts.

To determine whether this library could be screened to recover siRNA activity, 130 randomly chosen clones from E. coli containing the library were picked and pooled in groups of 10. These 13 pools were screened for their ability to inhibit PGL3 luciferase activity, as described in EXAMPLE 1. The library consisted of a maximum of 64 possible inserts, and only 2 of the pools from the 130 clones would be predicted to inhibit PGL3 luciferase activity. In agreement with this calculation, pools 8 and 11 had significant inhibitory activity, as shown in Table 5.

TABLE 5
Luciferase Assays to Measure Inhibition
by Partially Randomized PGL3 Specific Insert

| Plasmid or Pool Transfected (30 nanograms) | Luciferase activity (%) |
|---|---|
| pHippy | 100 |
| pHippyPGL3luc | 18 +/− 2 |
| Pool #1 | 101 +/− 11 |
| Pool #2 | 118 +/− 15 |
| Pool #3 | 80 +/− 8 |
| Pool #4 | 88 +/− 9 |
| Pool #5 | 131 +/− 16 |
| Pool #6 | 115 +/− 10 |
| Pool #7 | 102 +/− 9 |
| Pool #8 | 53 +/− 4 |
| Pool #9 | 114 +/− 13 |
| Pool #10 | 108 +/− 12 |
| Pool #11 | 31 +/− 2 |
| Pool #12 | 107 +/− 12 |
| Pool #13 | 96 +/− 9 |

These pools of 10 were further reduced to single clones, and each individual clone was rescreened for inhibitory activity. Pool 8 contained one clone that had inhibitory activity, and pool 11 contained two clones with inhibitory activity. Sequencing of these three clones revealed that these clones contained the original sequence against PGL3 (CTC). In contrast, 10 clones that did not inhibit PGL3 luciferase activity had random sequences at the mutant positions (clone 1: ACG, clone 2: AAG, clone 3: TCG, clone 4: GGG, clone 5, GCC, clone 6, CCG, clone 7, CCG, clone 8, TTG, clone 9, CCC, clone 10, GGT).

This set of experiments demonstrates that the pHippy system can be used for random siRNA screens. Specifically, libraries can be generated where all of the 21 nucleotides of the insert are random. This library would encompass multiple targets in every gene in the human genome and could be used for phenotypic single cell assays to identify genes required for the screened phenotype, without first knowing the siRNA sequence. For instance, a random insert library could be used to identify genes required for Wnt signaling by screening for siRNAs that inhibit the ability of Wnt to activate Super(8×)Topflash.

While the preferred embodiment of the invention has been illustrated and described, it will be appreciated that various changes can be made therein without departing from the spirit and scope of the invention.

1. An expression vector comprising:
(a) a first RNA polymerase III promoter operably associated with a first RNA polymerase III termination signal; and
(b) a second RNA polymerase III promoter operably associated with a second RNA polymerase III termination signal, wherein the first and second RNA polymerase III promoters are oriented to promote bidirectional transcription of an insert disposed between the first and the second RNA polymerase III termination signals.

2. The vector of claim 1 further comprising a cleavage site for a restriction enzyme disposed within each of the first and second RNA polymerase III termination signals.

3. The vector of claim 2 further comprising a recognition site for a restriction enzyme, wherein the cleavage site for the restriction enzyme is located outside the recognition site for the restriction enzyme.

4. The vector of claim 2, wherein the cleavage site is cleaved by a restriction enzyme selected from the group consisting of Alw1, Bbs1, Bbv1, BceA1, BciV1, BfuA1, Bmr1, Bpm1, BpuE1, Bsa1, BseR1, Bsg1, BsmA1, BsmB1, BsmF1, BspM1, Ear1, Eci1, Fau1, Fok1, Hga1, Hph1, MboII, Mly1, Mnl1, Ple1, Sap1, and SfaN1.

5. The vector of claim 4, wherein the restriction enzyme is BsmB1.

6. The vector of claim 1 further comprising an insert disposed between the first and second RNA polymerase III termination signals.

7. The vector of claim 6, wherein the size of the insert is between 19 and 29 nucleotides.

8. The vector of claim 6, wherein the size of the insert is between 19 and 23 nucleotides.

9. The vector of claim 1 further comprising a selectable marker operable in a eukaryotic cell.

10. The vector of claim 1 further comprising an origin of replication operable in a eukaryotic cell.

11. The vector of claim 1, wherein the vector is a plasmid vector.

12. The vector of claim 1, wherein the vector is a viral vector.

13. The vector of claim 1, wherein the vector is a linear vector.

14. A plurality of expression vectors, each comprising:
(a) a first RNA polymerase III promoter operably associated with a first RNA polymerase III termination signal;
(b) a second RNA polymerase III promoter operably associated with a second RNA polymerase III termination signal, wherein the first and second RNA polymerase III promoters are oriented to promote bidirectional transcription of an insert disposed between the first and the second RNA polymerase III termination signals; and
(c) an insert disposed between the first and second RNA polymerase III termination signals.

15. The plurality of vectors of claim 14, wherein the size of the insert is between 19 and 29 nucleotides.

16. The plurality of vectors of claim 14, wherein the size of the insert is between 19 and 23 nucleotides.

17. A method for inhibiting expression of a target gene, comprising introducing into a host cell an expression vector comprising:
(a) a first RNA polymerase III promoter operably associated with a first RNA polymerase III termination signal; and
(b) a second RNA polymerase III promoter operably associated with a second RNA polymerase III termination signal;
(c) a target gene-specific insert disposed between the first and the second RNA polymerase III termination signals, wherein the first and second RNA polymerase III promoters are oriented to promote bidirectional transcription of the target gene-specific insert to produce siRNA molecules that inhibit the expression of a target gene.

18. The method of claim 17, wherein the size of the target gene-specific insert is between 19 and 29 nucleotides.

19. The method of claim 17, wherein the size of the target gene-specific insert is between 19 and 23 nucleotides.

20. A method for determining the effect of an siRNA on a biological process, comprising the steps of:
(a) introducing into one or more host cells an expression vector comprising:
(i) a first RNA polymerase III promoter operably associated with a first RNA polymerase III termination signal;
(ii) a second RNA polymerase III promoter operably associated with a second RNA polymerase III termination signal; and
(iii) an insert disposed between the first and the second RNA polymerase III termination signals, wherein the first and second RNA polymerase III promoters are oriented to promote bidirectional transcription of the insert to produce siRNA molecules; and
(b) determining the effect of the siRNA molecules on a biological process of the one or more host cells.

21. The method of claim 20, wherein the size of the gene-specific insert is between 19 and 29 nucleotides.

22. The method of claim 20, wherein the size of the gene-specific insert is between 19 and 23 nucleotides.

23. The method of claim 20, wherein the effect on the biological process is determined using a reporter gene.

24. The method of claim 20, wherein the biological process mediates a biological signal transduction pathway.

25. The method of claim 20, wherein the biological process mediates the expression of a cell surface molecule.

26. The method of claim 20, wherein the biological process mediates stem cell differentiation.

27. The method of claim 20, wherein the insert comprises a random sequence of nucleotides.

28. The method of claim 20, wherein step (a) comprises introducing a plurality of expression vectors into one or more cells, wherein substantially all the vectors comprise a different insert.

29. The method of claim 28 further comprising the step of identifying at least one insert from which siRNA molecules are transcribed that produce the effect on the biological process.

30. A method for identifying an siRNA that affects a biological process, comprising the steps of:
(a) introducing a plurality of expression vectors comprising a plurality of inserts into one or more cells, wherein each of the plurality of expression vectors comprises:
(i) a first RNA polymerase III promoter operably associated with a first RNA polymerase III termination signal;
(ii) a second RNA polymerase III promoter operably associated with a second RNA polymerase III termination signal; and
(iii) an insert disposed between the first and the second RNA polymerase III termination signals, wherein the first and second RNA polymerase III promoters are oriented to promote bidirectional transcription of the insert to produce siRNA molecules; and
(b) identifying at least one insert from which siRNA molecules are transcribed that affect a biological process of the one or more cells.

31. The method of claim 30, wherein substantially all the expression vectors comprise a different insert.

32. The method of claim 30, wherein all the expression vectors comprise a different insert.

33. A kit for creating an expression vector for producing siRNA molecules, comprising:
(a) an expression vector comprising:
(i) a first RNA polymerase III promoter operably associated with a first RNA polymerase III termination signal;
(ii) a second RNA polymerase III promoter operably associated with a second RNA polymerase III termination signal; and
(iii) a restriction enzyme cleavage site disposed within each of the first and second RNA polymerase III termination signals, wherein the first and second RNA polymerase III promoters are oriented to promote bidirectional transcription of an insert introduced between the restriction enzyme cleavage sites; and
(b) packaging.

34. A kit for creating an expression vector for producing siRNA molecules, comprising:
(a) a first primer for amplifying a sense strand of a nucleic acid molecule comprising a first RNA polymerase III promoter operably associated with a first RNA polymerase III termination signal;
(b) a second primer for amplifying an antisense strand of a nucleic acid molecule comprising a second RNA polymerase III promoter operably associated with a second RNA polymerase III termination signal;
(c) a double-stranded nucleic acid template comprising the first RNA polymerase III promoter or the second RNA polymerase III promoter; and
(d) packaging.


SEQUENCE LISTING (32 sequences)

SEQ ID NO: 1 - DNA, 19 bp, Artificial Sequence
  Consensus Sequence for PSE
tnaccntnanynnaaagwg

SEQ ID NO: 2 - DNA, 97 bp, Artificial Sequence
  Modified HI Promoter
atttgcatgtcgctatgtgttctgggaaatcaccataaacgtgaaatgtctttggatttgggaatcttataagtggatcctgagaccgtctcaaaaa

SEQ ID NO: 3 - DNA, 333 bp, Artificial Sequence
  Modified U6 Promoter
ccccagtggaaagacgcgcaggcaaaacgcaccacgtgacggagcgtgaccgcgcgccgagcgcgcgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggctagcgtctcaaaaa

SEQ ID NO: 4 - DNA, 20 bp, Artificial Sequence
  U6p1 Primer
ccccagtggaaagacgcgca

SEQ ID NO: 5 - DNA, 41 bp, Artificial Sequence
  U6p2 Primer
tttttgagacgctagccacaagatatataaagccaagaaat

SEQ ID NO: 6 - DNA, 32 bp, Artificial Sequence
  H1not1 Primer
gaattcgcggccgcatttgcatgtcgctatgt

SEQ ID NO: 7 - DNA, 32 bp, Artificial Sequence
  U6mlu1 Primer
gaattcacgcgtccccagtggaaagacgcgca

SEQ ID NO: 8 - DNA, 41 bp, Artificial Sequence
  Pucori(S) Primer
gaattcacgcgtgcggccgcccactgagcgtcagaccccgt

SEQ ID NO: 9 - DNA, 27 bp, Artificial Sequence
  Pucori(AS) Primer
gaattcgccaggaaccgtaaaaaggcc

SEQ ID NO: 10 - DNA, 38 bp, Artificial Sequence
  SZPA (S) Primer
gaattcggatccacgcgtgaatgtgtgtcagttagggt

SEQ ID NO: 11 - DNA, 36 bp, Artificial Sequence
  SZPA(AS) Primer
gaattcggatccgagccccagacatgataagataca

SEQ ID NO: 12 - DNA, 23 bp, Artificial Sequence
  PGL3luciferase sense
aaaaggctcctcagaaacagctc

SEQ ID NO: 13 - DNA, 23 bp, Artificial Sequence
  PGL3luciferase antisense
aaaagagctgtttctgaggagcc

SEQ ID NO: 14 - DNA, 26 bp, Artificial Sequence
  EGFP sense
aaaagcaagctgaccctgaagttcat

SEQ ID NO: 15 - DNA, 26 bp, Artificial Sequence
  EGFP antisense
aaaaatgaacttcagggtcagcttgc

SEQ ID NO: 16 - DNA, 23 bp, Artificial Sequence
  LRP6#1sense
aaaaaggttcccttccacatcct

SEQ ID NO: 17 - DNA, 23 bp, Artificial Sequence
  LRP6#1 antisense
aaaaaggatgtggaagggaacct

SEQ ID NO: 18 - DNA, 26 bp, Artificial Sequence
  LRP6#2 sense
aaaaaaggttcccttccacatccttt

SEQ ID NO: 19 - DNA, 26 bp, Artificial Sequence
  LRP6#2 antisense
aaaaaaggatgtggaagggaaccttt

SEQ ID NO: 20 - DNA, 23 bp, Artificial Sequence
  LRP6#3 sense
aaaagaagatggcagccagggct

SEQ ID NO: 21 - DNA, 23 bp, Artificial Sequence
  LRP6#3 antisense
aaaaagccctggctgccatcttc

SEQ ID NO: 22 - DNA, 23 bp, Artificial Sequence
  LRP6#4 sense
aaaaggcacttacttccctgcaa

SEQ ID NO: 23 - DNA, 23 bp, Artificial Sequence
  LRP6#4 antisense
aaaattgcagggaagtaagtgcc

SEQ ID NO: 24 - DNA, 27 bp, Artificial Sequence
  LRP6#5 sense
aaaaaaggcacttacttccctgcaatt

SEQ ID NO: 25 - DNA, 27 bp, Artificial Sequence
  LRP6#5 antisense
aaaaaattgcagggaagtaagtgcctt

SEQ ID NO: 26 - DNA, 18 bp, Artificial Sequence
  U6PGL3p1 Primer
tggaaagacgcgcaggca

SEQ ID NO: 27 - DNA, 80 bp, Artificial Sequence
  U6PGL3p2 Primer
aaaaagagctgtttctgaggagcctctcttgaaggctcctcagaaacagctcggagatctttttgagacgctagccacaa

SEQ ID NO: 28 - DNA, 20 bp, Artificial Sequence
  H1PGL3p1 Primer
atttgcatgtcgctatgtgt

SEQ ID NO: 29 - DNA, 79 bp, Artificial Sequence
  H1PGL3p2 Primer
aaaaagagctgtttctgaggagcctctcttgaaggctcctcagaaacagctcggagatctttttgagacggtctcagga

SEQ ID NO: 30 - DNA, 97 bp, Artificial Sequence; identical to SEQ ID NO: 2
  H1p97 Primer

SEQ ID NO: 31 - DNA, 55 bp, Artificial Sequence
  H1-PGL3-U6 Primer
ctgagaccgtctcaaaaaggctcctcagaaacagctctttttgagacgctagcca

SEQ ID NO: 32 - DNA, 18 bp, Artificial Sequence
  U6p3 Primer
tggaaagacgcgcaggca